(19) United States
(12) Patent Application Publication
(10) Pub. No.: US 2026/0034290 A1
(43) Pub. Date: Feb. 5, 2026

(54) METHOD AND SYSTEM FOR FISTULA MONITORING

(71) Applicant: THE BOARD OF TRUSTEES OF THE UNIVERSITY OF ILLINOIS, Urbana, IL (US)

(72) Inventors: Richard Li, Saint Louis, MO (US); Eric Moran, Morgantown, WV (US); Szymon Kasperek, Decatur, IL (US); Rishab Rao Veldur, Chicago, IL (US); Satyansh Yeluri, Chicago, IL (US)

(73) Assignee: THE BOARD OF TRUSTEES OF THE UNIVERSITY OF ILLINOIS, Urbana, IL (US)

(21) Appl. No.: 19/284,231

(22) Filed: Jul. 29, 2025

Related U.S. Application Data
(60) Provisional application No. 63/677,264, filed on Jul. 30, 2024.

(51) Int. Cl. A61M 1/36; G16H 50/30

(52) U.S. Cl. CPC A61M 1/3656; A61M 1/3655; G16H 50/30; A61M 2205/18; A61M 2205/3368; A61M 2205/3375; A61M 2205/3561

(57) ABSTRACT

Aspects of the subject disclosure may include, for example, receiving acoustic signals captured by a sensor of a monitoring device, where the monitoring device is wearable by a patient and securely positioned on the patient's skin adjacent to an arteriovenous fistula (AVF); applying an algorithm to the acoustic signals to detect deviations from a baseline acoustic pattern, where the baseline acoustic pattern represents an expected operation of the AVF; and identifying potential patency-threatening events (PTEs) based on the deviations. Additional embodiments are disclosed.

### CROSS-REFERENCE TO RELATED APPLICATION(S)

The present application claims the benefit of priority to U.S. Provisional Application No. 63/677,264 filed Jul. 30, 2024, all sections of the aforementioned application(s) and/or patent(s) are incorporated herein by reference in their entirety.

### FIELD OF THE DISCLOSURE

The subject disclosure relates generally to method and system for fistula monitoring.

### BACKGROUND

Chronic kidney disease affects 32 million patients in the United States. When the kidneys are no longer able to filter blood, a patient is placed on dialysis. Dialysis is when a machine takes over the functions of your kidneys. In order for Dialysis to be performed an access method must be created by a surgeon.

Patients with end-stage renal disease often require hemodialysis, a process that necessitates reliable vascular access. Surgically created connections between an artery and a vein serve as access points for this procedure, which provide for the efficient extraction and return of blood during dialysis sessions.

An arteriovenous fistula (AVF) is a surgically created connection between an artery and a vein, which is typically in the arm of a patient. The purpose of an AVF is to provide a durable and reliable access point for the dialysis machine to extract and return blood at high volumes, whereby the high blood flow through the AVF helps to ensure that the dialysis process is efficient and effective.

In an AVF, high-pressure blood flow of the artery is directed into the vein, which causes the vein to enlarge and thicken over time. This process, known as maturation, makes the vein more robust and easier to access with dialysis needles. AVFs are preferred over other types of vascular access because they generally have lower complication rates and longer patency. However, AVFs are prone to complications such as stenosis (i.e., narrowing of the blood vessels) and thrombosis (i.e., clotting), which can lead to the failure of the fistula and the need for additional surgeries or interventions.

In current medical practices, the detection of when an AVF can first be utilized (i.e., maturation) involves a combination of clinical assessment and imaging techniques, which are time-consuming and inefficient. Vascular surgeons or nephrologists can perform a physical examination to assess the maturation of the AVF, which includes palpation to feel for the “thrill” (i.e., a vibration or buzzing sensation) and auscultation to listen for the “bruit” (i.e., a whooshing sound) using a stethoscope. These signs can indicate that blood is flowing through the fistula. A time-based assessment can be utilized since AVFs usually require a maturation period of 6 to 12 weeks after surgical creation before they can be used for dialysis. Duplex ultrasound can be performed which is an imaging technique used to evaluate the blood flow and structure of the AVF. Blood flow measurements can be taken (e.g., via Doppler ultrasound or other flow measurement devices). A trial cannulation (i.e., insertion of dialysis needle) may be performed to assess the AVF's readiness, which requires inserting a needle into the fistula to see if it can handle the blood flow required for dialysis without complications.

Unfortunately, these current processes of determining AVF readiness (i.e., maturation) are subjective and inefficient since they take time and cost expenditure.

In current medical practices, determining that an AVF can no longer be utilized for dialysis involves a combination of clinical assessment, patient symptoms, and diagnostic imaging, which are time-consuming and inefficient. Physical examinations as described above can be performed by the vascular surgeons or nephrologists to feel for the “thrill” and to listen for the “bruit” (a whooshing sound) using the stethoscope, whereby an absence or significant reduction of these signs may indicate that the AVF is no longer functional. Patient symptoms can be analyzed where patients are reporting symptoms such as swelling, pain, or redness around the AVF site, difficulty in achieving adequate blood flow during dialysis, or prolonged bleeding after needle removal, whereby these symptoms can be indicative of AVF dysfunction. Duplex ultrasound can be performed which can provide detailed information about the diameter of the vein, blood flow velocity, and the presence of any stenosis (narrowing) or thrombosis (clotting). Blood flow measurements can be taken whereby a significant reduction in blood flow rate can indicate that the AVF is no longer suitable for dialysis. An angiography can be performed which is an imaging technique that uses X-rays to view blood vessels) and may identify any blockages or abnormalities. A trial cannulation (i.e., insertion of dialysis needle) may be performed to assess the AVF's functionality.

Unfortunately, these current processes of determining AVF dysfunction are subjective and inefficient since they take time and cost expenditure.

### BRIEF DESCRIPTION OF THE DRAWINGS

Reference will now be made to the accompanying drawings, which are not necessarily drawn to scale, and wherein:

FIGS. 1 and 2 depict illustrative embodiments of systems that provide monitoring of an AVF;

FIGS. 3-6 depict an illustrative embodiment of a monitoring device that can be used in monitoring of an AVF;

FIG. 7 depicts an illustrative embodiment of a user interface that can be used in monitoring of an AVF;

FIG. 8 depicts an illustrative method that can be used in monitoring of an AVF;

FIG. 9 depicts an illustrative diagrammatic representation of a machine in the form of a computer system within which a set of instructions, when executed, may cause the machine to perform any one or more of the methodologies disclosed herein;

FIGS. 10-11 depict illustrative embodiments of monitoring devices that can be used in monitoring of an AVF; and

FIGS. 12-13 illustrate Mel frequency spectrograms for a failed AVF and for a healthy AVF.

### DETAILED DESCRIPTION

In one or more embodiments, monitoring of AVFs (e.g., continuously, periodically, near-real-time and/or real-time) can be provided using a wearable device that captures acoustic signals. For example, the wearable device can utilize one or more microphones to detect sounds associated with the AVF including detecting and/or determining characteristic sounds of a functioning fistula, such as an audible thrill. In one or more embodiments, the device and methodology described herein can leverage a “venous hum”, which is an auditory and vibrational signal produced by blood flow through the fistula, as an input for monitoring.

In one or more embodiments, Artificial Intelligence (AI) and/or Machine Learning (ML) algorithms (e.g., modeling) can be employed to analyze sounds associated with the AVF for deviations from a baseline. In one or more embodiments, alerts can be generated and transmitted, such as to the patient and/or clinician, as to potential issues, such as stenosis or thrombosis, enabling timely intervention. In one or more embodiments, in contrast to existing practices, a non-invasive monitoring (e.g., continuously, periodically, near-real-time and/or real-time) approach can be implemented which can predict fistula failure before it occurs, thereby improving patient outcomes and reducing healthcare costs.

In one or more embodiments, continuous outpatient monitoring is provided. Unlike current practices requiring periodic doctor visits and manual palpation, one or more embodiments allow for continuously, periodically, near-real-time and/or real-time monitoring of AVFs in any environment, including at home. This can be particularly beneficial for late-stage kidney disease patients undergoing frequent hemodialysis sessions.

In one or more embodiments, the venous hum can be utilized as a primary input for monitoring. For instance, the venous hum can provide or allow determining a consistent and unbiased measure of blood flow through the fistula.

In one or more embodiments, AI/ML can be employed to analyze the venous hum and detect deviations from the norm. This can provide for early detection of stenosis and thrombosis, which are common causes of fistula failure. In one or more embodiments, AI/ML models can be refined based on patient-specific data, improving accuracy over time.

In one or more embodiments, the wearable device can be of a practical size and shape to facilitate its use and make it more comfortable for the patient. For instance, a non-invasive, peel-and-stick patch can be used which is positioned on top of the location of the AVF. In other embodiments, the patch can be placed on top of an adhesive. In one or more embodiments, the wearable device does not require surgical implantation and/or a complex setup process.

In one or more embodiments, smartphone integration can be provided. For example, the wearable device can pair with or otherwise communicate with a smartphone app that receives collected data from the wearable device and that allows for tracking a fistula status over time. Other functionality can include providing warnings for patency-threatening events (PTEs) (e.g., caused by or potentially indicative of stenosis, thrombosis, infection, aneurysm formation, mechanical compression, and so forth) and/or interfacing with clinicians, clinics, hospitals or other health-care providers to send alerts (e.g., real-time or near-real-time) for remote medical monitoring.

In one or more embodiments, the system and methods allow for an early intervention and improved outcomes for patients. For example, by providing monitoring (e.g., continuously, periodically, near-real-time and/or real-time) and early detection of potential issues, the wearable device enables timely medical intervention, which can improve salvage of failing fistulas, reduce the need for additional surgeries, and ultimately enhance patient outcomes and quality of life.

In one or more embodiments, an objective assessment of the AVF is provided, which can improve the accuracy and timing of AVF utilization for dialysis (e.g., an earlier detection of maturation).

In one or more embodiments, an objective assessment of the AVF is provided, which can improve the accuracy and timing of detecting and/or predicting AVF dysfunction, and which can enable timely medical intervention.

One embodiment of the subject disclosure entails a method for monitoring of an arteriovenous fistula (AVF) in a patient. The method includes receiving, by a processing system including a processor, acoustic signals captured by a sensor of a monitoring device, where the monitoring device is wearable by the patient and securely positioned on the patient's skin adjacent to the AVF. The method includes applying, by the processing system, an algorithm to the acoustic signals to detect deviations from a baseline acoustic pattern, where the baseline acoustic pattern represents operation of the AVF after maturation. The method includes identifying, by the processing system, potential patency-threatening events (PTEs) based on the deviations.

Another embodiment of the subject disclosure entails a monitoring device for monitoring of an arteriovenous fistula (AVF) in a patient. The monitoring device includes a housing configured to be applied to skin of the patient adjacent to the AVF; a microphone configured to capture venous hum signals produced by blood flow through the AVF; and a communications component configured to transmit the captured venous hum signals to a communication device. Transmitting of the captured venous hum signals to the communication device causes the communication device to apply a machine learning algorithm to the venous hum signals to: determine a maturation of the AVF, generate a baseline acoustic signal that represents normal patency of the AVF, or detect a potential Patency-Threatening event (PTE) according to a deviation between the venous hum signals and the baseline acoustic signal.

Yet another embodiment of the subject disclosure entails a non-transitory machine-readable medium, comprising executable instructions that, when executed by a processing system including a processor, facilitate performance of operations. The operations include receiving acoustic signals captured by a sensor of a monitoring device, where the monitoring device is wearable by a patient and securely positioned on the patient's skin adjacent to an arteriovenous fistula (AVF); applying an algorithm to the acoustic signals to detect deviations from a baseline acoustic pattern, where the baseline acoustic pattern represents an expected operation of the AVF; and identifying potential patency-threatening events (PTEs) based on the deviations.

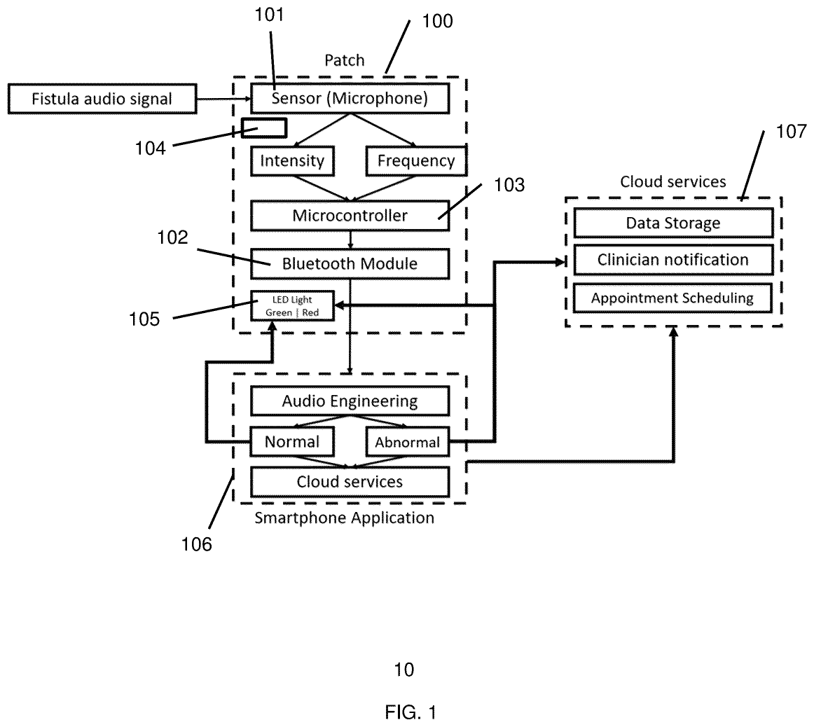

Referring to FIG. 1, a system 10 is illustrated which provides for monitoring of an AVF (not shown) through use of AVF monitoring device (e.g., wearable device) 100. As an example, the monitoring device 100 can include a number of components, circuits, hardware and/or software that facilitates accurate monitoring (e.g., continuously, periodically, near-real-time and/or real-time) of the AVF. In one embodiment, the device 100 can be configured to be applied to the skin adjacent to the AVF, capturing acoustic or venous hum signals produced by blood flow through the AVF. In other embodiments, other techniques and structure can be used to enable wearing of the monitoring device 100 such as a strap or band. The device 100 can include one or more sensors 101, a communications module 102 (e.g., Bluetooth), a microcontroller 103, a power source 104 (e.g., a battery), and an indicator 105 (e.g., an LED). In one embodiment, the device 100 can communicate with one or more communication devices (e.g., a smartphone) 106 and one or more cloud-based servers 107 to process and store data.

In one or more embodiments, the sensor 101 can include one or more acoustic sensors (e.g., a microphone(s)) that capture audio signals including venous hum signals produced by blood flow through the AVF. In one embodiment. the sensor 101 can include multiple microphones which are configured to facilitate capturing the venous hum signals as acoustic data, such as a first microphone facing the AVF for capturing the venous hum and a second microphone facing the environment (e.g., in an opposite direction as to the AVF to capture environmental sounds which can be utilized for noise filtering, noise cancellation and/or weighing the accuracy of the captured signal). For example, the use of multiple microphones can enhance the accuracy and reliability of signal capture by providing a more comprehensive acoustic profile of the AVF. The sensor 101 can be positioned near the AVF including at a particular angle and/or distance (which can include adjacent to or in close proximity to the patient's skin) to ensure optimal or improved signal capture. The captured audio signals can then be provided to the microcontroller 103 for further processing. The sensor 101 can be highly sensitive, ensuring accurate detection of the venous hum signals. In another embodiment, the sensor 101 can be a single microphone.

In one embodiment in addition to the microphone(s), the sensor 101 can also include or can be used in conjunction with one or more temperature sensors. As an example, temperature sensors can be configured to monitor the temperature of the skin adjacent to the AVF. Temperature monitoring can provide additional diagnostic information, as changes in temperature may indicate inflammation or infection at the AVF site. In one embodiment, the temperature sensors can be positioned at opposite ends of the AVF. In another embodiment, the temperature sensors can obtain environmental temperatures which can also be analyzed to determine effects on the AVF and/or effects on the accuracy of monitoring AVF patency via acoustical signals. The temperature data can be provided to the microcontroller 103 and/or to other devices such as the smartphone 106 for analysis in conjunction with the venous hum signals. Other types of sensors can be utilized with monitoring device 100 which are integrated with the device or in communication with the device, such as blood flow sensor, blood oxygen detector, blood pressure, patient mobility, heart rate, and so forth.

The communication module 102 can be of various types (e.g., Bluetooth) that enable wireless communications and that can be integrated into the device 100 to facilitate wireless communication between the device 100 and the smartphone 106. For example, the Bluetooth module 102 can transmit captured venous hum signals, temperature data and/or other collected data from the device 100 (e.g., one or more sensors 101) to the smartphone 106, such as in near-real-time or real-time. This wireless communication enables monitoring (e.g., continuously, periodically, near-real-time and/or real-time) without the need for physical connections, enhancing the convenience and usability of the device 100. The Bluetooth module 102 can ensure that the data is transmitted securely and efficiently to the smartphone 106 for further analysis. The microcontroller 103 can operate as a central processing unit of the device 100, which can manage, process and/or distribute the data captured by the device (e.g., one or more sensors 101) and transmitted by the Bluetooth module 102.

In one embodiment, an AI/ML model or algorithm can be applied to collected venous hum signals to determine and generate a baseline acoustic signal. This baseline acoustic signal can represent the patency of the AVF. For example to determine maturation of the AVF, the monitoring device 100, equipped with the microphone(s) 101, can capture venous hum signals produced by blood flow through the AVF where the data collection can begin at the time of AVF creation and continues throughout and beyond the maturation period.

The raw venous hum signals can be preprocessed to remove noise and artifacts. This may involve filtering techniques to isolate the relevant frequency range and normalize the signal amplitude. The preprocessing step can provide for input data that is clean and consistent for further analysis. The preprocessed venous hum signals can be analyzed to extract key features that characterize the acoustic properties of the AVF. These features may include frequency components, amplitude variations, temporal patterns, or other characteristics that can be analyzed.

In one embodiment, Mel frequency diagrams or images can be generated which can be analyzed to determine AVF operation (e.g., maturation, normal operation after maturation, potential failure, failure, etc.). For example, a convolutional neural network (CNN) algorithm can be applied to the Mel frequency diagrams for analyzing acoustic signals including determining deviations from a baseline and/or determining when an AVF has matured. Feature extraction can facilitate reducing the dimensionality of the data while preserving the essential information needed for classification. An AI/ML algorithm, such as CNN, can be trained using the extracted features from the venous hum signals. The training process can include feeding the algorithm with labeled data, where the stages of AVF maturation are known (e.g., via another detection method such as ultrasound or imaging). The algorithm learns to recognize the patterns and characteristics associated with different stages of AVF maturation from this training data.

In one embodiment, once the AI/ML model is trained, it can be used to generate a baseline acoustic signal that represents the normal patency of the AVF, such as at various stages of maturation. For instance, the baseline acoustic signal can serve as a reference point for continuously, periodically, near-real-time and/or real-time monitoring. It can encapsulate typical frequency, amplitude, and temporal patterns of a healthy AVF at different maturation stages. In one embodiment, different baseline signals can be utilized according to other known factors that can effect acoustic signal analysis, such as blood pressure, blood flow rate, body temperature, room temperature, and so forth. In one embodiment, the baseline signal can represent the expected acoustic pattern that is determined for an AVF that is matured.

In one embodiment, the monitoring device 100 can continuously, periodically, near-real-time and/or real-time capture venous hum signals and transmit them to the smartphone 106 so that the AI/ML algorithm can compare the incoming signals to the baseline acoustic signals corresponding to different maturation stages. For example, any deviations from the expected patterns can be analyzed to assess the maturation progress of the AVF.

In one embodiment, the AI/ML algorithm can detect maturation of the AVF by identifying when the venous hum signals match or are within a threshold of baseline acoustic signals associated with a fully matured AVF (e.g., confirmed for this particular AVF via another procedure such as duplex ultrasound, determined according to one or more other AVFs via another procedure such as duplex ultrasound, determined according to ML/AI models using acoustic signals collected for other AVFs confirmed to be mature, and so forth). This can include recognizing the characteristic frequency, amplitude, and/or temporal patterns that indicate the AVF has reached a stage suitable for dialysis.

In one embodiment, when the AI/ML algorithm detects that the AVF has matured, it triggers real-time alerts to equipment of the patient and healthcare providers, such as via a smartphone application. For instance, these alerts can inform the patient and clinicians that the AVF is ready for use in dialysis.

In one embodiment, the AI/ML algorithm can be refined (e.g., continuously or frequently) based on the stored venous hum data and detected maturation stages of the AVF. This iterative learning process can improve the accuracy of maturation detection and can enhance the ability of the AI/ML modeling to adapt to individual patient characteristics and variations in AVF maturation.

In one embodiment, the monitoring device 100 can be positioned initially with a patient and cross-referenced with a duplex ultrasound to establish a baseline acoustic signal.

For example, by leveraging AI/ML, the monitoring device 100 can accurately detect the maturation of the AVF, providing timely information to patients and healthcare providers. This approach allows for the AVF to be used for dialysis at an optimal time, improving patient outcomes and reducing the risk of complications associated with premature or delayed use.

In one embodiment, the smartphone 106 (or other communication device(s)) can receive information (e.g., venous hum signals, temperature data, processed raw sensor data, and so forth) from the device 100 via the Bluetooth module 102. In one embodiment, the smartphone 106 can have or can access an application that processes the venous hum signals using the AI/ML described herein to generate a baseline acoustic signal. In one embodiment, the smartphone 106 can continuously, periodically, near-real-time and/or real-time monitor the venous hum signals and can compare them to the baseline acoustic signal to detect deviations indicative of PTEs. In one embodiment, the smartphone 106 can also analyze the temperature data to detect any abnormal temperature changes. The smartphone 106 can provide real-time alerts to the patient and healthcare providers when a PTE or abnormal temperature change is detected. The smartphone 106 may also display a visual representation of the venous hum signals, temperature data, and/or detected PTEs, enhancing patient engagement and understanding of their AVF status.

In one or more embodiments, the AI/ML algorithm can operate in the smartphone 106 or in another device (e.g., the Cloud) that is accessible to the smartphone. In this example, the monitoring device 100 captures the venous hum signals and transmits them to the smartphone 106 via a communication connection. A smartphone application can then process these signals using the AI/ML algorithm to generate a baseline acoustic signal, then continuously, periodically, near-real-time and/or real-time monitor for deviations, and then detect PTEs. This setup leverages the processing power and storage capabilities of the smartphone 106, allowing the monitoring device 100 to remain compact and energy-efficient.

However, in other embodiments, the AI/ML algorithm can operate in either or both of the monitoring device or the smartphone (or another device including in the Cloud). For example, where the monitoring device 100 is equipped with sufficient processing power and storage, the AI/ML algorithm can be implemented directly within the device, which would enable on-device processing and reduce the need for continuous data transmission to the smartphone 106. In one or more embodiments, the microcontroller 103 can monitor (e.g., continuously, periodically, near-real-time and/or real-time) the venous hum signals and can compare them to the baseline acoustic signal to detect deviations indicative of patency-threatening events (PTEs) such as stenosis and thrombosis. In one embodiment, the microcontroller 103 can analyze temperature data to detect any abnormal temperature changes that may indicate inflammation or infection. In one embodiment, the microcontroller 103 can initiate or facilitate refining the AI/ML algorithm based on collected and/or stored data to improve the accuracy of PTE detection over time.

In other embodiments, a hybrid approach could be used, where initial signal processing occurs in the monitoring device 100, and more complex analysis and AI/ML operations are performed on the smartphone 106 or in the cloud. The flexibility in the placement of the AI/ML algorithm allows for various design configurations, each with its own advantages in terms of power consumption, processing speed, and data management.

A battery 104 can power the device 100, ensuring continuous operation. Although other power sources can be utilized. The battery 104 can be rechargeable, providing power to the sensor(s) 101, Bluetooth module 102, and microcontroller 103. The battery 104 ensures that the device 100 can operate continuously for a particular amount of time, such as up to 14 days (or other time periods) before needing replacement or recharging. The battery 104 can be integrated into the device 100 in a manner that maintains the compact and lightweight design of the device 100, ensuring patient comfort and ease of use.

The indicator 105 can provide feedback to a patient regarding AVF and/or device status. For example, an LED indicator 105 can provide immediate visual feedback to the patient regarding the status of the AVF, such as through the LED indicator changing color to signal operation and/or the detection of a PTE. For instance, the LED indicator 105 may shine green to indicate operation and red to indicate the detection of a PTE. This visual feedback mechanism can enhance patient safety by providing real-time alerts, allowing for rapid clinical response and potentially preventing complications.

The server 107 (which is described herein as being cloud-based but could be other types of servers including a healthcare provider server that provides AVF monitoring services) can store the processed venous hum data, baseline acoustic signal, temperature data, other collected data, and/or detected PTEs for continuously, periodically, near-real-time and/or real-time monitoring and analysis. The cloud-based server 107 can provide remote access to healthcare providers, allowing them to monitor the patient's AVF status and make informed clinical decisions. In one embodiment, the cloud-based server 107 can also support the refinement of the machine learning algorithm based on the stored data, improving the accuracy of PTE detection over time. This centralized data storage can facilitate long-term tracking of the AVF's status and can support remote monitoring by healthcare providers. In one embodiment, the server 107 can be utilized to assist in training of the AI/ML modeling, such as through sharing acoustic data (e.g., securely and/or anonymously) across multiple patients that provides for better model training and a more accurate application of the AI/ML model to identify AVF maturation, PTEs, AVF failure, AVF potential failure, predictions as to an AVF failure (which may or may not include a time frame for the predicted failure), and so forth.

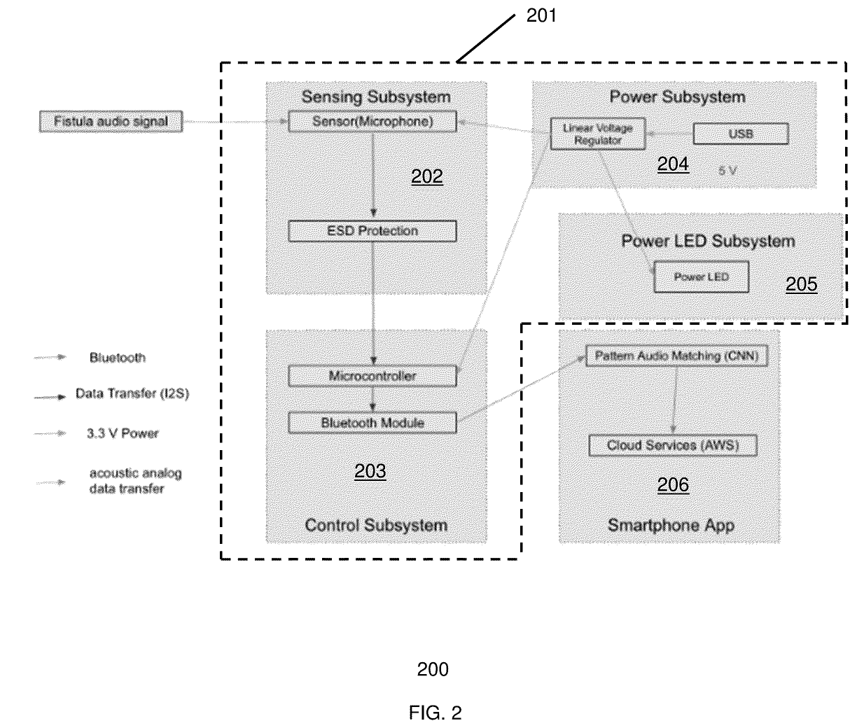

FIG. 2 shows a system 200 for continuously, periodically, near-real-time and/or real-time monitoring of an AVF. The system 200 includes a monitoring device 201 and a smartphone application 206. The monitoring device 201 comprises a sensing subsystem 202, a control subsystem 203, a power subsystem 204, and a power LED subsystem 205. The monitoring device 201 captures venous hum signals produced by blood flow through the AVF and processes these signals to detect a maturation of the AVF and/or PTEs.

The sensing subsystem 202 can include a sensor(s) or microphone(s) configured to capture venous hum signals. In one embodiment, the sensing subsystem 202 can include electrostatic discharge (ESD) protection to safeguard the sensor and other components from electrical damage.

The control subsystem 203 can include a microcontroller and a communication component such as a Bluetooth module. The microcontroller receives the venous hum signals from the sensing subsystem 202 and processes these signals. In one embodiment, the Bluetooth module transmits the processed signals to the smartphone application 206 for further analysis. In one embodiment, the control subsystem 203 can include or have access to pattern audio matching, such as using a convolutional neural network, and/or cloud services for data storage and analysis.

The power subsystem 204 can include a linear voltage regulator and a USB interface. The linear voltage regulator ensures a stable power supply to the monitoring device 201, while the USB interface allows for recharging the device's battery. The power subsystem 204 can provide a requisite amount of power (e.g., a 3.3 V power supply) to the sensing subsystem 202 and the control subsystem 203.

The power LED subsystem 205 can include a power LED that provides visual feedback to the patient. The power LED indicates the operational status of the monitoring device 201 and/or alerts the patient when a PTE or potential PTE is detected. The power LED subsystem 205 is powered by the power subsystem 204.

The monitoring device 201 can communicate with the smartphone application 203 via the Bluetooth module in the control subsystem 203. The smartphone application 206 processes the venous hum signals using an AI/ML algorithm to determine maturation of the AVF and/or to generate a baseline acoustic signal for the AVF. In one embodiment, the application 206 continuously or frequently monitors the venous hum signals and compares them to the baseline acoustic signal to detect deviations indicative of PTEs or potential PTEs. The processed data can be stored in cloud services for continuously, periodically, near-real-time and/or real-time monitoring and analysis, allowing healthcare providers to remotely access the patient's AVF status.

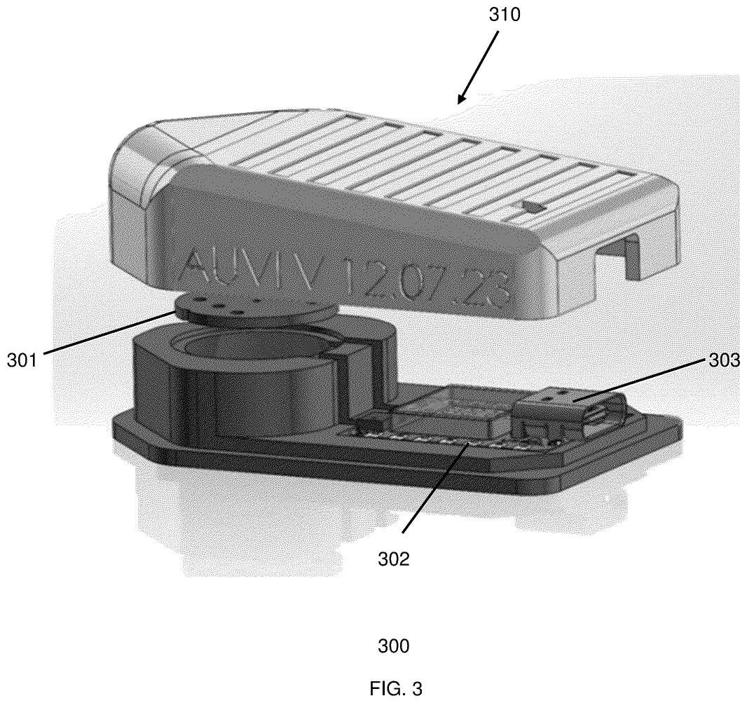

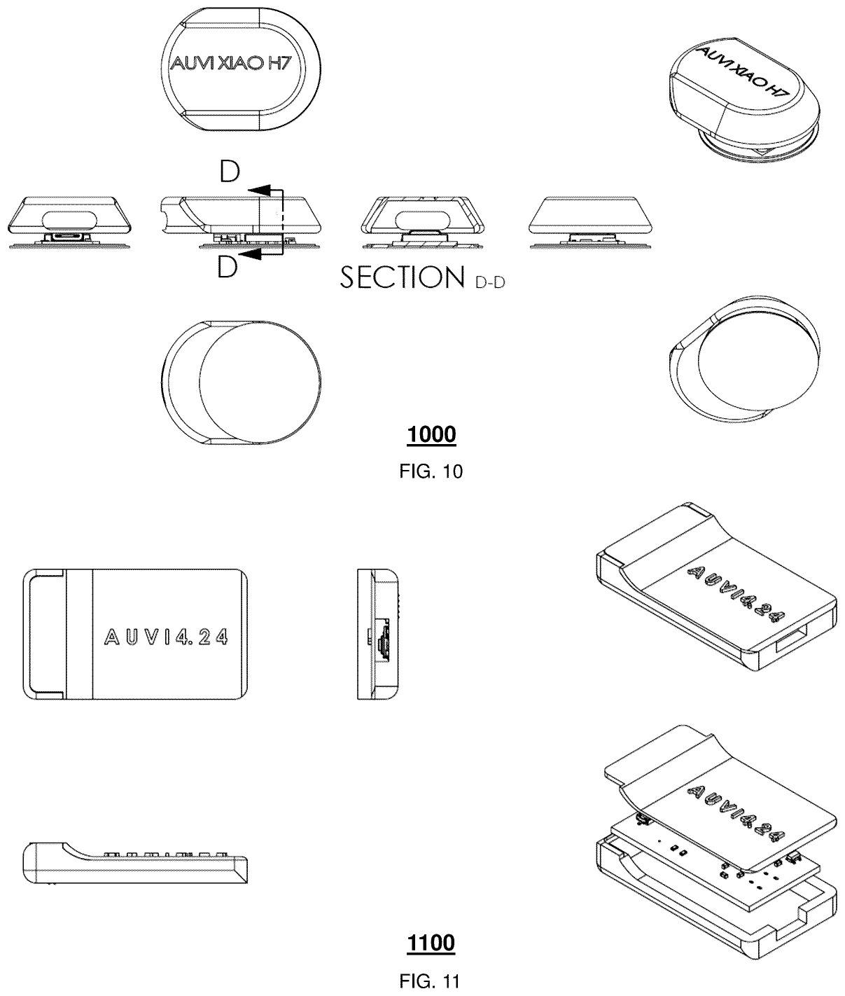

FIGS. 3-6 illustrate an embodiment of an AVF monitoring device 300. It should be understood that the AVF monitoring devices described herein can have various shapes, sizes, components and/or functionality which may or may not be illustrated in FIGS. 3-6. As an example, other embodiments of monitoring devices 1000, 1100 are shown in the exploded views depicted in FIGS. 10-11, which can include other shapes, dimensions, component positioning, functionality, sensing components/techniques, communication components/techniques, and so forth. Monitoring devices 1000, 1100 can perform one, some or all of the functions described throughout the various embodiments herein and/or can include one, some or all of the components described throughout the various embodiments herein.

FIG. 3 illustrates an exploded view of an AVF monitoring device 300. The device 300 can include a housing 310, a microcontroller 302, a USB-C port 303, and a microphone 301. The housing 310 can encase the internal components, providing protection and structural support. The microcontroller 302 can process the acoustic signals including venous hum signals captured by the microphone 301 and can transmit the data to a smartphone application. The USB-C port 303 allows for wired data transfer and/or charging of the device.

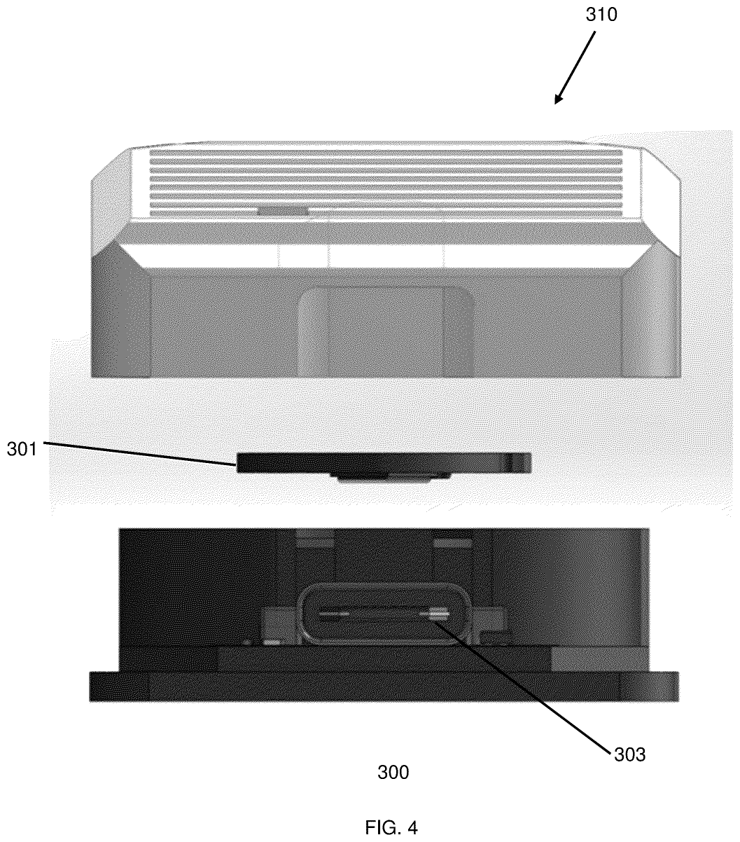

FIG. 4 illustrates a side exploded view of the AVF monitoring device 300, highlighting the microphone 301. The housing 310 can be compact and ergonomic, ensuring that the device can be comfortably worn by the patient. The USB-C port 303 can be positioned for easy access, allowing for convenient charging and/or data transfer. The microphone 301 can be strategically placed to capture venous hum signals accurately.

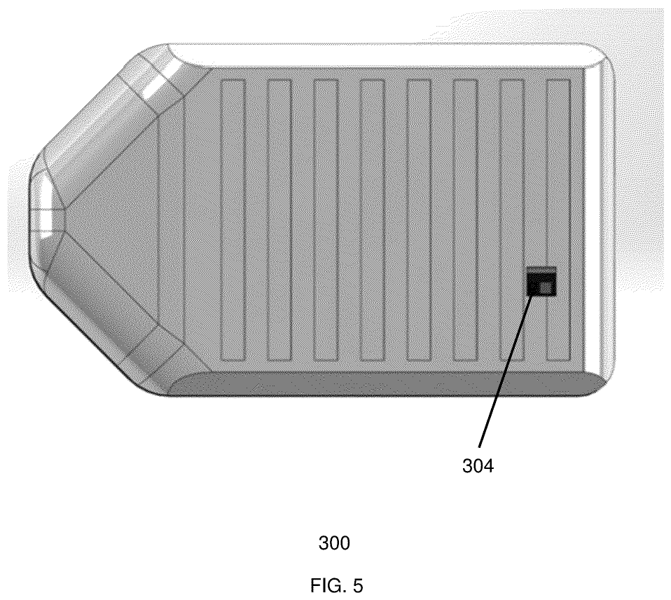

FIG. 5 shows a top view of the AVF monitoring device 300, with a focus on the LED indicator 304. The LED indicator 304 provides visual feedback to the patient, changing color to signal the operational status of the device and/or the detection of any PTEs or other undesired conditions.

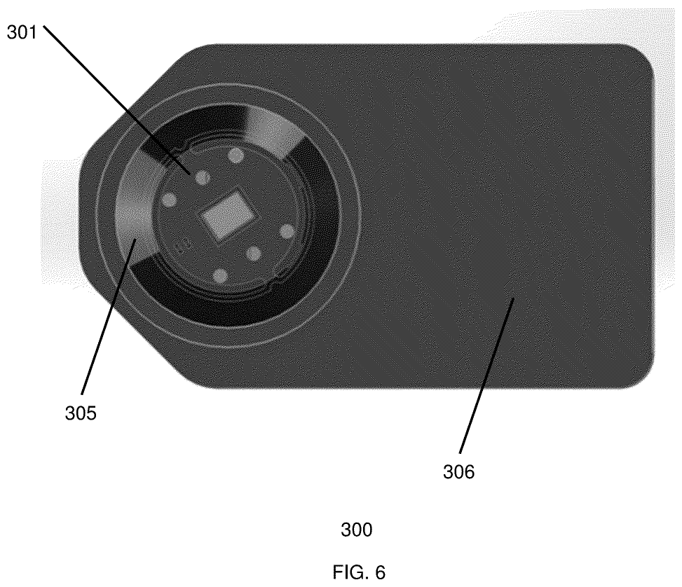

FIG. 6 shows a bottom view of the AVF monitoring device 300, highlighting the microphone 301, and the opening 305 in the base 306. In one embodiment, an adhesive layer can be positioned on some or all of the base 306 to enable the device 300 to be worn by the patient. The microphone 301 can be positioned in the opening 305 (which can have various shapes and sizes including a tapered or conical shape) and is configured to capture venous hum signals produced by blood flow through the AVF. The opening 305 can work in conjunction with the microphone 301 to enhance signal detection, such as allowing the microphone to be adjacent to and/or abutting the skin of the patient. The adhesive layer on the base 306 allows the device to be securely attached to the patient's skin adjacent to the AVF. The design can enable the microphone 301 to maintain consistent contact with the skin, providing accurate and reliable signal capture.

The monitoring device 300 can include other features that enhance the functionality, usability, and adaptability of the device for different patient needs and clinical scenarios. For example, the device 300 can include an integrated display. For instance, the monitoring device 300 can include an integrated display on the housing 310. The display would provide real-time information about the AVF status, including visual alerts for PTEs including descriptions or codes identifying the particular PTE such as stenosis and thrombosis. This would allow patients to receive immediate feedback without needing to check their smartphone, enhancing convenience and accessibility.

As another example, device 300 can include multiple sensors of a same or different type. For example, additional sensors can be integrated into or in communication with the monitoring device 300, such as pressure sensor(s) and flow sensor(s), in conjunction with the microphone(s) and/or temperature sensor(s). The multi-sensor device 300 would provide a more comprehensive assessment of the AVF's condition by measuring various physiological parameters. The data from these sensors can be combined and analyzed, such as using the AI/ML modeling, to improve the accuracy of PTE detection/prediction and AVF maturation assessment.

In one embodiment, the AVF monitoring device can be an implantable device. For example, the AVF monitoring device can have a size and shape (and made of appropriate material) so that it operates as an implantable device that can be surgically placed adjacent to the AVF. In this embodiment, the implantable device continuously, periodically, near-real-time and/or real-time monitors the venous hum signals and other physiological parameters, transmitting the data wirelessly to an external receiver or smartphone. This can be particularly suitable for patients who require long-term monitoring and prefer a more discreet solution.

In one embodiment, the AVF monitoring device can include an extended battery life which optimizes power consumption of the monitoring device to extend its battery life. For example, it can include energy-efficient components and power management algorithms that allow the device to operate continuously for extended periods, such as up to 30 days (or other time periods), before needing recharging or replacement. This can reduce the frequency of maintenance and enhance the device's usability for patients with limited mobility or access to charging facilities.

In one embodiment, the AVF monitoring device can provide other components or techniques for wearability. For example, the monitoring device can be integrated into common wearable items such as wristbands, armbands, smartwatches, and so forth. The wearable integration provides a seamless and comfortable monitoring experience for patients, allowing them to carry out their daily activities without interruption. The wearable device can continuously or frequently capture and transmit venous hum signals to the smartphone application for analysis.

In one embodiment, cloud-based analytics can be used by the monitoring system. For example, cloud-based analytics can be leveraged to enhance processing and storage capabilities of the monitoring system. The venous hum signals and other sensor data can be transmitted to a cloud server(s), where AI/ML algorithms analyze the data in real-time. The cloud-based approach allows for more complex and resource-intensive computations, improving the accuracy and reliability of PTE detection/prediction and AVF maturation assessment. Additionally, healthcare providers can access the cloud-based analytics platform to monitor multiple patients remotely and make informed clinical decisions.

In one embodiment, the AVF monitoring device can include a metal plate or other material for vibration or improving sensitivity and acoustic collection. For example, a metal plate within the monitoring device (e.g., near the microphone and/or between the microphone and the AVF) can act as a resonator, amplifying the vibrations produced by blood flow through the AVF. This would improve the sensitivity and accuracy of the microphone in capturing the venous hum signals, especially in cases where the signals are weak or difficult to detect.

In one embodiment, the AVF monitoring device can include a rigid base such as at the bottom of the housing or near the center (or where the microphone is located) of the bottom of the housing. For example, the rigid base would provide structural support and stability to the monitoring device, ensuring that it remains securely positioned on the patient's skin adjacent to the AVF. This would help maintain consistent contact between the device and the skin, improving the reliability of signal capture and reducing the risk of displacement during patient movement.

In one embodiment, the AVF monitoring device can include adjustable features for positioning the microphone(s). For example, structure can be added that allows for re-orientation and/or repositioning of the microphone(s) to allow for precise positioning of the microphone(s). This would enable customization of the device's configuration based on the patient's anatomy and the specific location of the AVF. Adjustable microphone positioning would ensure optimal signal capture and enhance the overall performance of the monitoring device.

In one embodiment, the AVF monitoring system can utilize Retrieval-Augmented Generation (RAG) to improve the output of a large language model used for detecting the deviations of the acoustic signals from the baseline and/or detecting maturation of the AVF. The RAG, which can be used in a number of different ways including in conjunction with AI/ML modeling described herein, can reference an authoritative knowledge base outside of the training data sources used for the AI/ML modeling before generating a response. In one embodiment, the AVF monitoring system can employ RAG for providing medical diagnosis assistance in conjunction with the PTE data provided by the AVF monitoring device. By rapidly accessing and integrating information from medical literature, patient records, and research papers, RAG Architecture can assist healthcare professionals in expediting diagnosis and improving patient outcomes.

In one embodiment, the AVF monitoring system can employ RAG for healthcare information retrieval. For example, RAG can assist healthcare professionals in accessing essential information from electronic health records, medical texts, and clinical guidelines. By simplifying access to vital information, it can facilitate more informed clinical decisions, and promotes evidence-based treatment.

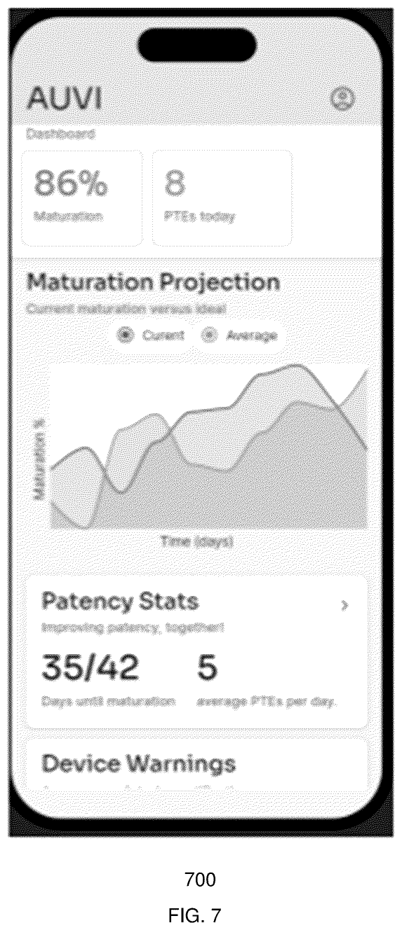

FIG. 7 illustrates a user interface or dashboard 700 that can be viewed by patients, clinicians, healthcare providers or other persons/entities. As an example, UI 700 can provide progression of the fistula's patency, and/or can provide feedback on events that threaten the fistula (lying on it, sleeping on it, etc.). In one embodiment, UI 700 allows for patient's to interact and understand their fistula.

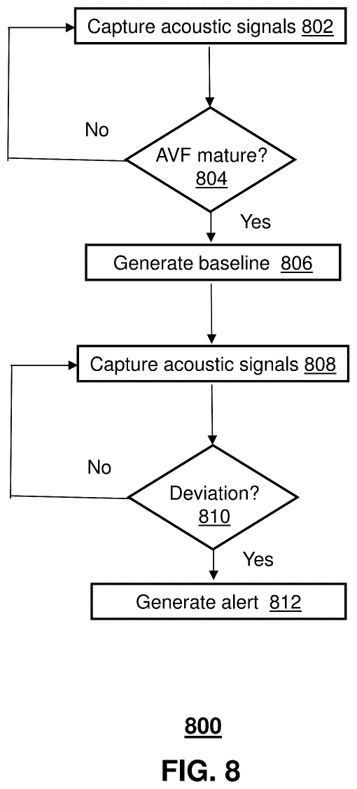

FIG. 8 illustrates a method 800 for monitoring an AVF to detect a maturation and to detect or predict PTEs or other undesired conditions associated with the AVF. At 802, acoustic signals can be captured via a sensor of a monitoring device, wear the monitoring device is wearable by a patient or otherwise securely positioned on the patient's skin adjacent to an AVF. This monitoring can be done at 804 to determine a maturation of the AVF according to the acoustic signals captured by the sensor of the monitoring device. This can be done in a number of different ways including comparing the acoustic signals to known acoustic signals of matured AVFs utilizing AI/ML models.

At 806, a baseline signal can be generated (e.g., from acoustic signals once the AVF is matured). At 808, acoustic signals can be captured via the sensor of the monitoring device (e.g., after the maturation of the AVF). This monitoring can be done at 810 to determine any potential PTEs, PTEs or other undesired conditions with respect to the AVF. The monitoring can include comparing the acoustic signals captured by the sensor of the monitoring device (at 810) to the baseline signal to detect any deviations. This can be done in a number of different ways including comparing the acoustic signals to baseline signal utilizing AI/ML models. In one embodiment, the AI/ML modeling can detect the deviation(s) and identify the potential PTEs, PTEs or other undesired conditions with respect to the AVF. In one embodiment, the AI/ML modeling can detect the deviation(s) and generate or otherwise determine predictions as to any potential PTEs, PTEs or other undesired conditions with respect to the AVF. For instance, these predictions can include time as to when it will occur (including ranges), extent of the PTE (e.g., how much the diameter of the AVF will decrease), and so forth. In one or more embodiments, the baseline signal (or acoustic pattern) can represent an expected operation of the AVF.

At 812, alerts can be generated according to the monitoring. For example, an alert can be transmitted to a smartphone of the patient. As another example, an alert can be transmitted to equipment of a clinician. These alerts can include various information that is discerned from the AVF monitoring an can be provided to various devices of various persons/entities. In one embodiment, the AI/ML algorithm or modeling is a CNN.

From the foregoing descriptions, it would be evident to an artisan with ordinary skill in the art that the aforementioned embodiments can be modified, reduced, or enhanced without departing from the scope and spirit of the claims described below.

In one or more embodiments, by continuously, periodically, near-real-time and/or real-time monitoring the venous hum signals and comparing them to the baseline acoustic signal, the monitoring device can detect deviations indicative of PTEs, such as stenosis and thrombosis, and can provide for the potential issues being identified promptly, enabling timely medical intervention.

In one or more embodiments, real-time alerts can be provided to the patient and healthcare providers via the smartphone application when a PTE is detected or predicted. This immediate feedback mechanism can enhance patient safety and allow for rapid clinical response, potentially preventing complications and improving patient outcomes.

In one or more embodiments, the processed venous hum data, baseline acoustic signal, and detected/predicted PTEs can be stored in a server (e.g., a cloud-based server) for continuously, periodically, near-real-time and/or real-time monitoring and analysis, which facilitates long-term tracking of the AVF's status and supports remote monitoring by healthcare providers.

In one or more embodiments, the AI/ML can be refined based on the stored data, improving the accuracy of PTE detection over time. For example, this iterative learning process can ensure that the monitoring system becomes more effective with continued use, adapting to individual patient characteristics and enhancing overall reliability.

In one embodiment, the signal input is an auditory/vibrational signal from the blood flowing through the graft—a “venous hum”. In one embodiment, the device would train to a “normal” venous hum at the time of fistula introduction, and then would detect any deviations from this norm. In one embodiment, after training on the normalized dataset, the system refines to the patient specific venous hum. In one embodiment, a deviation in signal arising from increased oscillations in the pressure waveform can be indicative of stenosis. In one embodiment, a lack of detected waveform would be indicative of thrombosis.

In one embodiment, a smartphone app can pair with the device to track fistula status over time. As an example, the app provides warning and tracking of PTEs, including warnings regarding the fistula viability. In one embodiment, when interfacing with clinics/hospitals, the system can send information about PTEs in real time. In one embodiment, the App can track the time to maturation of the fistula, which is the time before the fistula would be established and ready for use with hemodialysis. In one embodiment, the system and/or App can track hemodialysis sessions and scheduling. In one embodiment, the system can have a machine learning component, where the device “learns” the patient's normal venous hum and subsequent monitoring is based on that training data.

In one or more embodiments, the baseline acoustic signal can be obtained at various times after maturation of the AVF, such as one week after it has matured. In other embodiments, the baseline acoustic signal can be updated, such as when the AVF has been confirmed as operating properly.

In one or more embodiments, venous hum signals can be captured from numerous AVFs of numerous patients and used as training data. In one embodiment, the training data can include acoustic signals that are paired with known AVF status, such as not yet matured, matured and operating as expected, and/or failed. In one embodiment, the training data can include acoustic signals that are paired with known AVF status and with other data associated with the patients and/or the environments when the acoustic signals are being captured, such as biometric data of the patients (e.g., blood pressure, blood flow, heart rate, temperature data, patient size, patient age, dimensions of the AVF, changes to dimensions of the vein and artery associated with the AVF, etc.) and/or environmental conditions (e.g., outdoor temperature, outdoor noise, patient's activity such as sleeping or walking, etc.).

In one embodiment, the monitoring system can capture or discern various parameters associated with the acoustic signals including intensity, waveform and/or frequency.

In one embodiment, the housing of the monitoring device can be made of a plastic or other material that provides protection for the inner components but is comfortable for the patient.

In one embodiment, the AI/ML modeling can detect deviations of the acoustic signals from the baseline and determine or predict an abnormal flow condition for the AVF. In one embodiment, the monitoring device can capture acoustic signals during dialysis. These acoustic signals can be analyzed by the ML/AI modeling (e.g., trained on data that was captured during various dialysis procedures of the same and/or different patients) for making determinations and predictions with respect to the dialysis procedure.

In one embodiment, the monitoring device can include or have access to position and/or motion sensors, such as a GPS and/or an accelerometer, for collecting various information with respect to the patient including whether the patient is walking or sleeping (e.g., which could lead to different acoustic signals), whether the patient is in a place where there is too much noise or is quiet enough for accurate acoustic signal capture, and so forth.

In one embodiment, the monitoring device can include or have access to a pressure sensor, such as to determine if the patient is leaning against the device (e.g., during sleeping) which could effect the device and/or effect blood flow through the AVF.

In one embodiment, detected patient activity can be utilized for assisting

the ML/AI modeling in detecting a deviation from the baseline. For instance, acoustic signals captured when a patient is running may be different from acoustic signals captured when a patient is sleeping, and the AI/ML modeling can learn the distinctions. In one embodiment, different baselines can be generated and utilized for different conditions of the patient, such as sleeping vs. sitting vs running.

In one embodiment, ambient noise may be detected (e.g., by a second microphone of the monitoring device pointed away from the AVF) and the detection of the ambient noise can be used for noise filtering, flagging the accuracy of any deviation determination, and/or not using the particular captured acoustic signals for deviation analysis by the AI/ML modeling.

In one embodiment, various data processing and transformation can be applied to the acoustic signal data to facilitate the maturation analysis and/or the deviation analysis, including fourier transforms.

In one embodiment, the monitoring device can include a hollowed-out area (e.g., a circular opening) of various shapes where the microphone is positioned to facilitate acoustic signal capture. In one embodiment, the hollowed-out area can include a ledge or other structure to allow the microphone to be secured to or supported by the housing of the monitoring device. In one embodiment, the monitoring device can include a diaphragm where the microphone is positioned. The diaphragm can have various shapes including a conical shape away from the AVF (e.g., a larger diameter of the diaphragm abutting the patient's skin). In one embodiment, the monitoring device can include acoustic amplification components and/or apply acoustic amplification techniques to increase the sensitivity with respect to capturing the acoustic signals of the AVF blood flow.

In one embodiment, the monitoring device can include an activation string, plastic tab or arming pin that can be manipulated or pulled to activate the battery power supply (e.g., remove any separation of contacts).

In one embodiment, the monitoring device can include multiple microphones that are facing the AVF for capturing acoustic signals. In one embodiment, the monitoring device can include temperature sensors, such as opposite ends of the AVF, which can measure temperature differential for discerning blood flow or other temperature=related information. In one embodiment, the monitoring device can include a PCB that is flexible, such as enabling curvature to match the shape of the patient's arm or other location of the AVF. In one embodiment, the monitoring system can apply different AI/ML modeling to determine different results, such as a first AI/ML modeling to detect a deviation of an acoustic signal from the baseline, and applying a second AI/ML modeling to determine blood flow rate through the AVF or other temperature dependent biometrics of the patient, such as based on a temperature differential, environmental temperature effecting the deviation analysis accuracy (e.g., calculating an error range), and so forth.

In one embodiment, the monitoring device can include a connection mechanism, such as adhesive tape, that can be replaced so that the monitoring device can be re-secured to the skin after a time period of use. In one embodiment, the monitoring device can include a sensor that detects a distance between the microphone and the patient's skin and/or detects when the microphone is not in contact with the patient's skin (e.g., a pressure sensor, capacitance sensor, ultrasonic sensor, laser sensor, and so forth). In one embodiment, portions of the PCB are rigid while other portions are not, such as the microphone and the housing near the CPU being rigid.

In one embodiment, the monitoring device can position the microphone in various areas, such as in the center of the device or along the side of the device.

In one embodiment, the monitoring device can accommodate different patients and different AVF positions through an adjustable orientation that enables shifting the angle of the microphone (e.g., via a pivot mechanism) and/or its position (e.g., via a sliding mechanism) with respect to the monitoring device so as to better capture acoustic signals from the AVF.

In one embodiment, the acoustic signals can be captured for a particular length of time (e.g., 5 seconds or some other time period which, for example, may be larger than a heart beat rate) and/or can be sliced or otherwise broken into smaller portions to facilitate AI/ML analysis.

In one embodiment, the analysis of the acoustic signals can be done using CNN applied to Mel-frequency diagrams and/or applying other AI/ML modeling that is image-based from spectrograms or other images discerned from the acoustic signals. In one embodiment, the analysis of the acoustic signals can focus on (at least in part) concentration of frequencies. In one embodiment, the analysis of the acoustic signals can apply particular thresholds, such as frequency threshold or threshold range or dB threshold or threshold range. In one embodiment, the analysis of the acoustic signals utilizes a Support Vector Machine algorithm, such as having audio data as the input.

The utilization of this technology can span various sectors, with a significant emphasis on healthcare applications. Specifically, one or more of the embodiments can be instrumental in the monitoring of AV fistulas, heart sounds, and lung sounds. Further application of the system can be to diseases including those currently diagnosed or monitored using audio data, which would include conditions generally associated with the cardiac, pulmonary, or gastric system, including asthma, sleep apnea, COPD, lung cancer, cardiac monitoring, other vascular functions, and bowel obstruction. The ability to monitor these conditions remotely, and flag abnormalities for intervention can provide expeditious intervention and improve patient outcomes.

Additionally, one or more of the embodiments can extend into industrial domains. Continuously, periodically, near-real-time and/or real-time monitoring devices find application in overseeing machinery operations within manufacturing facilities or detecting irregularities in the functioning of equipment requiring moving parts. The nature of the monitoring system, with its functions of monitoring and flagging abnormal acoustic signals, would allow for application also in these domains. Applications outside of healthcare in areas such as aeronautics or manufacturing, or in any area where a primary change in acoustic signal indicates a problem, are areas in which one or more of the embodiments is applicable.

In one or more embodiments, the monitoring device can be a wearable noninvasive remote monitoring device that sits adjacent to the AV fistula. It can monitor the fistula for indications of a failed or upcoming AV fistula failure. By detecting fistula failure early, medical teams can salvage the fistulas preventing surgeries and increasing patient outcomes. In a healthy AV fistula, blood flow from the artery to the vein creates a turbulent flow, which produces a characteristic sound known as the bruit. This sound is heard as a continuous, whooshing noise. The presence and quality of this bruit are indicators of blood flow through the fistula.

In one or more embodiments, the monitoring device can listen to this sound and determine the patency of the fistula. As an example, an evaluation of the turbulence of the blood flow through the fistula can be performed. A healthy fistula will have a characteristic laminar flow and produce a whooshing sound that caretakers are thought to look for. In a stenosed fistula, the diameter of the blood vessel will decrease. By conservation of mass and momentum the blood flow should greatly speed up. Blood can be assumed to be an incompressible fluid. In one or more embodiments, the Reynolds number can be used to reveal whether flow is laminar or turbulent:

NR=2*p*v*r*n

For NR<2000 flow is laminar

For NR>3000 flow is turbulent

Using the doubling of fluid speed with the reduction of diameter that is assumed when using the equations above, in one or more embodiments, it can be determined that the flow through the fistula will quickly become turbulent. This turbulent flow produces a characteristic high pitch sound that the monitoring device aims to detect and ultimately predict through subtle changes over time.

In one or more embodiments, a detection algorithm can be used to create a binary classification and ultimately a prediction on whether a fistula has failed or is failing. As an example, in order to make a classification on abnormal fistula audio first a standard analog signal processing model is used to screen for high frequency data that signals a failing fistula.

In one or more embodiments, AI/ML can be employed, such as CNN, where Mel frequency spectrograms are generated from the audio signals passed through a Fourier transform. In one or more embodiments, metadata about the surgical site can be fed into the model. The CNN can consist of multiple convolutional layers.

In one or more embodiments, Vision Transformers (ViT) can be applied, where Mel frequency spectrograms are generated from the audio signals passed through a Fourier transform. In one or more embodiments, metadata about the surgical site can be fed into the model. The model can be trained with Adaptive Moment Estimation optimizer (ADAM), which is an optimization algorithm used in deep learning. In one or more embodiments, other stochastic gradient descent (SGD) algorithms can be used or other algorithms that update the weights of a neural network during training.

In one or more embodiments, SVM can be applied that is trained on pure audio data classification to make a binary classification.

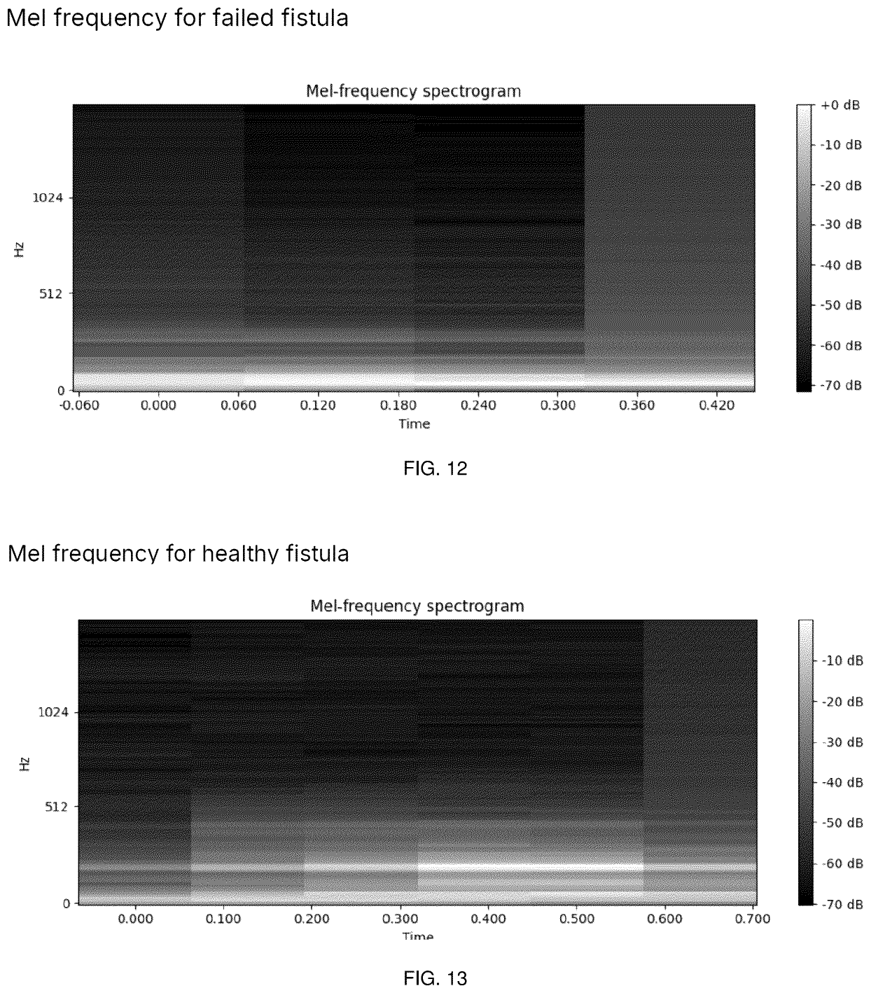

Referring to FIGS. 12-13, Mel frequency spectrograms are shown for a failed AVF (FIG. 12) and for a healthy AVF (FIG. 13). It should be understood that these spectrograms are examples and other Mel frequency spectrograms generated according to the exemplary embodiments described herein can be different, including having different values for frequency, time and decibel level.

In one or more embodiments, the monitoring system can include a patch, a phone app, a clinical dashboard, and cloud services. For example, the patch can include a microphone(s) placed against the skin to hear the sounds produced by blood flowing through the fistula. For example, the microphone has a target signal to noise ratio of 60 db, although other ratios can be utilized. One example of such a microphone is an invenSense ICS4343. For instance, the microphone can listen for change in blood flow as described herein.

In one or more embodiments, the monitoring device can have material placed between the skin and microphone and/or can detect vibration coming from a plate or other material adjacent to the skin. In one or more embodiments, two or more microphones or other sensors placed around the skin to take audio or other sensor readings from multiple locations. In one or more embodiments, the monitoring device can utilize millimeter wave technology.

In one or more embodiments, the monitoring device can include semi-invasive parts. In one or more embodiments, the monitoring device can be fully invasive and positioned inside the fistula (in whole or in part such as a sensor within the fistula) or positioned next to the fistula (in whole or in part such as a sensor within the arm adjacent to the fistula).

In one or more embodiments, the monitoring device can include a System on a Chip (SOC) (or other integrate circuit technologies) with integrated flash memory and a built in antenna with wireless (e.g., Bluetooth) low energy capabilities, such as an Espressif esp32 C3.

In one or more embodiments, the monitoring device can have an external facing microphone to identify and/or remove background noise. In one or more embodiments, the monitoring device can have multiple temperature sensors (e.g., two temperature sensors) placed along the fistula to measure blood flow (e.g., via temperature differential). For instance, both sensors can be placed along fistula in regions exposed to blood flow. One sensor can be placed in a region with stable body temperature and one sensor placed in a region with temperature affected with blood flow:

delta(t)=temperature(upstream)−temperature (downstream)

Q=M*C*delta(t)

m=pv

V=Q/(p*c*delta(t))

with standard k empirical calibration after. If blood flow decreases or increases above a certain threshold, then in one or more embodiments the monitoring device can generate an alarm.

In one or more embodiments, the monitoring device can include a pressure sensor to measure how deep the fistula is positioned. In one or more embodiments, the monitoring device can include a light sensor (e.g. a green light sensor) to take blood flow measurements. In one or more embodiments, the monitoring device can include sensors for water detection, temperature, and/or pressure of the internal device. In one or more embodiments, the monitoring device can include an imu/gyroscope and accelerometer, such as to control or otherwise limit the taking of measurements only when the monitoring device is sitting still.

In one or more embodiments, the monitoring device such as a patch can include medical tape that encompasses the entire or a portion of the bottom surface of the device. In one or more embodiments, the monitoring device/patch can contain a hole in a specific region to expose the microphone to the skin. In one or more embodiments, the monitoring device can have a housing which may be rigid plastic or flexible material. In one or more embodiments, the monitoring device's PCB itself can be entirely flexible or can be partially flexible and rigid in certain regions around the microphone and SOC. In one or more embodiments, the monitoring device's housing is designed to minimize snagging with a low profile and no sharp edges. In one or more embodiments, the monitoring device can have a “beetle shape” and/or an oval shape. In one or more embodiments, the monitoring device can include a cellular chip to connect directly to the cloud bypassing the need for a smartphone app or other communication device that is in proximity/short range to the monitoring device.

In one or more embodiments, the monitoring device is usable for a month (although other longer or shorter time frames can exist) and/or the tape or other securing mechanism can last two weeks (although other longer or shorter time frames can exist) and can have a holding mechanism (e.g., plastic) to swap tape but not the device.

In one or more embodiments, the monitoring device can have an arming tab that when pulled causes the monitoring device to activate and turned on, such as permanently. In one or more embodiments, the monitoring device can be reclaimed with the shell swapped and internal circuitry reused.

In one or more embodiments, the monitoring device can have the capability to shift the microphone in different positions and/or shift other sensors to provide customizability for each patient. This can be done using a number of different structures or techniques including slideable connections, locking mechanisms, pivots, etc.

In one or more embodiments, the monitoring device can be moved around the surgical site to prevent skin breakdown. In one or more embodiments, the monitoring device can include a vibration motor that alerts the patient when the device is not able to get a valid reading, the fistula has or is predicted to fail, and/or to provide a haptic alert for other reasons. In one or more embodiments, the monitoring device can include a LED status indicator.

In one or more embodiments, the monitoring system can include or otherwise make available an application, such as a smartphone App. For example, the App can be developed using a React-Native framework allowing for simultaneous development for both IOS and Android devices, although other development schemes can be employed.

In one or more embodiments, the monitoring system can use a smartphone's Bluetooth Low Energy (BLE) capabilities to establish a secure connection with the monitoring device. In one or more embodiments, the monitoring system's smartphone App. can poll the monitoring device for audio samples throughout the day. For example, the polling can be according to a schedule which may be evenly distributed over time or unevenly distributed and/or the polling can be in response to events or other determined information, such as detecting a blood flow rate change using a blood flow rate sensor or detecting a position change of the fistula using a pressure sensor. In one embodiment, a combination of scheduled polling and event-driven polling can be employed.

In one or more embodiments, the monitoring system can use a smartphone App to send data to cloud services (or other server(s)) for processing and storage, such as via an API (e.g., AWS API). In one or more embodiments, the monitoring system can use a smartphone App to connect directly to healthcare provider software (e.g., Epic) or other hospital dashboards currently in use. In one or more embodiments, the monitoring system can use a smartphone App to display or otherwise present fistula maturation information, failure detections, and/or predicted projections (including predicted maturation and/or predicted failures).

In one or more embodiments, the monitoring system can connect (e.g., via the smartphone APP) to a clinical dashboard(s), which can further allow patients to make or view upcoming appointments. In one or more embodiments, the monitoring system can use a smartphone App to provide reminders to replace the monitoring device and/or provide other communication(s) from healthcare providers. In one or more embodiments, the monitoring system can use a smartphone App to indicate or display one or more device statistics, such as battery life and other warnings generated by the monitoring device.

In one or more embodiments, the monitoring system (directly and/or via the smartphone App) can provide feedback on user movement and/or events that may be threatening the fistula. In one or more embodiments, the monitoring system (directly and/or via the smartphone App) can provide feedback on user movement and/or events that may threaten or compromise the monitoring device (e.g., sleeping on it, usage in water, moving it off position, etc.). In one or more embodiments, the monitoring system (directly and/or via the smartphone App) can provide resources for understanding fistula and proper care of it. In one or more embodiments, the monitoring system (directly and/or via the smartphone App) can provide emergency notifications regarding the status of the fistula.

In one or more embodiments, the monitoring system can provide a dashboard such as an online website, which can be developed in a number of different ways using the Javascript React framework. In one or more embodiments, the monitoring system website can allow healthcare providers to securely logon to the dashboard and monitor patient information. In one or more embodiments, the monitoring system can allow the dashboard to connect to a database (e.g., an AWS Database) to retrieve information about fistula progression and audio samples.

In one or more embodiments, the monitoring system can provide a dashboard that allows healthcare providers to contact patients, send reminders, set up visits, or otherwise interact and manage the fistula. In one or more embodiments, the monitoring system allows healthcare providers (directly and/or via the smartphone App) to use the dashboard to real-time activate the patient's monitoring device and/or get an up-to-date audio sample and analysis of the fistula.

In one or more embodiments, the monitoring system (directly and/or via the smartphone App) can utilize cloud services, such as AWS S3, which can be used to store all audio samples for a patient. In one or more embodiments, the monitoring system (directly and/or via the smartphone App) can utilize AWS DynamoDB for storage and retrieval of patient information for user and doctors.

In one or more embodiments, the monitoring system (directly and/or via the smartphone App) can utilize AWS Lambda which uses cloud resources for audio processing of fistula audio. In one or more embodiments, the monitoring system (directly and/or via the smartphone App) can use AI/ML algorithms coded through Python. In one or more embodiments, the monitoring system (directly and/or via the smartphone App) can use a AI/ML model trained on data samples to predict maturation of fistulas and provide warnings to healthcare providers/doctors and patients. In one or more embodiments, the monitoring system (directly and/or via the smartphone App) can utilize AWS Eventbridge such as to trigger notifications for patients and doctors based on the outcomes of audio processing or any other database changes that require immediate alerts. In one or more embodiments, the monitoring system (directly and/or via the smartphone App) can utilize Amazon Elastic Container Registry to store/maintain training model and/or python code used for the processing of audio samples. In one or more embodiments, the monitoring system (directly and/or via the smartphone App) can utilize Amazon SageMaker which can operate as a comprehensive service for building, training, and deploying machine learning models. In one or more embodiments, the monitoring system (directly and/or via the smartphone App) can utilize AWS Identity and Access Management (IAM) to manage access and permissions for all users of the smartphone App and dashboard.

In one or more embodiments, the monitoring system (directly and/or via the smartphone App) can utilize Terraform to define, provision, and manage cloud infrastructure resources for quick development of the cloud services being used.

Other suitable modifications can be applied to the subject disclosure. Accordingly, the reader is directed to the claims for a fuller understanding of the breadth and scope of the subject disclosure.

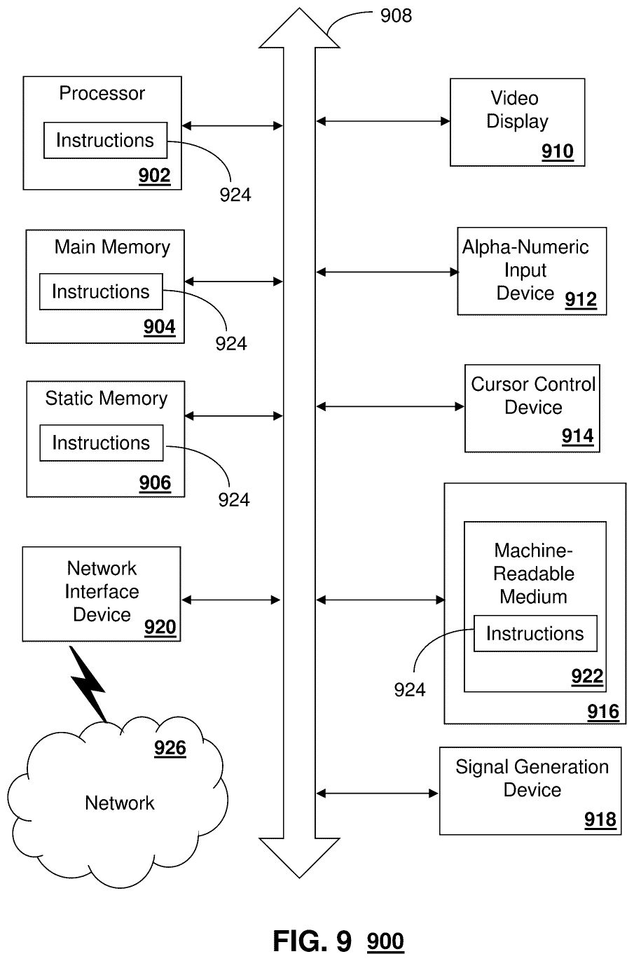

FIG. 9 depicts an exemplary diagrammatic representation of a machine in the form of a computer system 900 within which a set of instructions, when executed, may cause the machine to perform any one or more of the methods discussed above. In some embodiments, the machine may be connected (e.g., using a network) to other machines. In a networked deployment, the machine may operate in the capacity of a server or a client user machine in server-client user network environment, or as a peer machine in a peer-to-peer (or distributed) network environment.

The machine may comprise a server computer, a client user computer, a personal computer (PC), a tablet PC, a smart phone, a laptop computer, a desktop computer, a control system, a network router, switch or bridge, or any machine capable of executing a set of instructions (sequential or otherwise) that specify actions to be taken by that machine. It will be understood that a communication device of the subject disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methods discussed herein.

The computer system 900 may include a processor 902 (e.g., a central processing unit (CPU), a graphics processing unit (GPU, or both), a main memory 904 and a static memory 906, which communicate with each other via a bus 908. The computer system 900 may further include a video display unit 910 (e.g., a liquid crystal display (LCD), a flat panel, or a solid state display. The computer system 900 may include an input device 912 (e.g., a keyboard), a cursor control device 914 (e.g., a mouse), a disk drive unit 916, a signal generation device 918 (e.g., a speaker or remote control) and a network interface device 920.

The disk drive unit 916 may include a tangible computer-readable storage medium 922 on which is stored one or more sets of instructions (e.g., software 924) embodying any one or more of the methods or functions described herein, including those methods illustrated above. The instructions 924 may also reside, completely or at least partially, within the main memory 904, the static memory 906, and/or within the processor 902 during execution thereof by the computer system 900. The main memory 904 and the processor 902 also may constitute tangible computer-readable storage media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the subject disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

While the tangible computer-readable storage medium 922 is shown in an example embodiment to be a single medium, the term “tangible computer-readable storage medium” should be taken to include a single medium or multiple media (e.g., a centralized or distributed database, and/or associated caches and servers) that store the one or more sets of instructions. The term “tangible computer-readable storage medium” shall also be taken to include any non-transitory medium that is capable of storing or encoding a set of instructions for execution by the machine and that cause the machine to perform any one or more of the methods of the subject disclosure.

The term “tangible computer-readable storage medium” shall accordingly be taken to include, but not be limited to: solid-state memories such as a memory card or other package that houses one or more read-only (non-volatile) memories, random access memories, or other re-writable (volatile) memories, a magneto-optical or optical medium such as a disk or tape, or other tangible media which can be used to store information. Accordingly, the disclosure is considered to include any one or more of a tangible computer-readable storage medium, as listed herein and including art-recognized equivalents and successor media, in which the software implementations herein are stored.

Although the present specification describes components and functions implemented in the embodiments with reference to particular standards and protocols, the disclosure is not limited to such standards and protocols. Each of the standards for Internet and other packet switched network transmission (e.g., TCP/IP, UDP/IP, HTML, HTTP) represent examples of the state of the art. Such standards are from time-to-time superseded by faster or more efficient equivalents having essentially the same functions. Wireless standards for device detection (e.g., RFID), short-range communications (e.g., Bluetooth, WiFi, Zigbee), and long-range communications (e.g., WiMAX, GSM, CDMA) are contemplated for use by computer system 300.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

The Abstract of the Disclosure is provided with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

What is claimed is:

1. A method for monitoring of an arteriovenous fistula (AVF) in a patient, the method comprising:
receiving, by a processing system including a processor, acoustic signals captured by a sensor of a monitoring device, the monitoring device being wearable by the patient and securely positioned on skin of the patient adjacent to the AVF;
applying, by the processing system, an algorithm to the acoustic signals to detect deviations from a baseline acoustic pattern, wherein the baseline acoustic pattern represents operation of the AVF after maturation; and
identifying, by the processing system, potential patency-threatening events (PTEs) based on the deviations.

2. The method of claim 1, further comprising:
providing an alert via a smartphone application when a potential PTE is identified.

3. The method of claim 1, further comprising:
storing the acoustic signals and the potential PTE in a server resulting in stored data.

4. The method of claim 1, wherein the algorithm is an artificial intelligence/machine learning (AI/ML) algorithm, and further comprising:
refining the AI/ML based on the stored data to improve an accuracy of PTE detection over time.

5. The method of claim 1, wherein the receiving of the acoustic signals is via a wireless communication.

6. The method of claim 1, wherein the sensor is a microphone configured to capture venous hum signals as acoustic data.

7. The method of claim 1, wherein the algorithm is a convolutional neural network (CNN).

8. The method of claim 2, wherein the smartphone application is further configured to display a visual representation of patency status.

9. The method of claim 1, further comprising:
receiving temperature data associated with a temperature of the skin adjacent to the AVF captured by a temperature sensor of the monitoring device; and
analyzing the temperature data to detect any abnormal temperature changes that may indicate inflammation or infection.

10. The method of claim 1, further comprising:
determining, by the processing system, a maturation of the AVF according to prior acoustic signals captured by the sensor of the monitoring device.

11. A monitoring device for monitoring of an arteriovenous fistula (AVF) in a patient, the monitoring device comprising:
a housing configured to be applied to skin of the patient adjacent to the AVF;
a microphone configured to capture venous hum signals produced by blood flow through the AVF; and
a communications component configured to transmit the captured venous hum signals to a communication device,
wherein transmitting of the captured venous hum signals to the communication device causes the communication device to apply a machine learning algorithm to the venous hum signals to: determine a maturation of the AVF, generate a baseline acoustic signal that represents normal patency of the AVF, or detect a potential Patency-Threatening event (PTE) according to a deviation between the venous hum signals and the baseline acoustic signal.

12. The monitoring device of claim 11, wherein the housing is configured as a peel-and-stick patch using a biocompatible adhesive.

13. The monitoring device of claim 11, further comprising a battery configured to power the microphone and the communications component.

14. The monitoring device of claim 11, further comprising an LED indicator configured to provide a visual alert to the patient when the potential PTE is detected.

15. The monitoring device of claim 11, further comprising a temperature sensor configured to monitor the temperature of the skin adjacent to the AVF, wherein temperature data is transmitted by the communications component to the communication device.

16. A non-transitory machine-readable medium, comprising executable instructions that, when executed by a processing system including a processor, facilitate performance of operations, the operations comprising:
receiving acoustic signals captured by a sensor of a monitoring device, the monitoring device being wearable by a patient and securely positioned on the patient's skin adjacent to an arteriovenous fistula (AVF);
applying an algorithm to the acoustic signals to detect deviations from a baseline acoustic pattern, wherein the baseline acoustic pattern represents an expected operation of the AVF; and
identifying potential patency-threatening events (PTEs) based on the deviations.

17. The non-transitory machine-readable medium of claim 16, wherein the algorithm is a convolutional neural network (CNN).

18. The non-transitory machine-readable medium of claim 16, wherein the operations further comprise transmitting an alert to a smartphone of the patient.

19. The non-transitory machine-readable medium of claim 16, wherein the operations further comprise transmitting an alert to equipment of a clinician.

20. The non-transitory machine-readable medium of claim 16, wherein the operations further comprise determining a maturation of the AVF according to prior acoustic signals captured by the sensor of the monitoring device.
